(19) United States
(12) Patent Application Publication
(10) Pub. No.: US 2025/0122454 A1
(43) Pub. Date: Apr. 17, 2025

(54) PHOTOBIOREACTOR SYSTEM FOR THE REDUCTION OF AMMONIA AND CARBON DIOXIDE GASES IN POULTRY HOUSES

(71) Applicants: BURSA ULUDAG ÜNIVERSITESI, Nilüfer, Bursa (TR); BURSA TEKNIK ÜNIVERSITESI REKTÖRLÜGÜ, Yildirim, Bursa (TR)

(72) Inventors: Seyit UGUZ, Nilüfer, Bursa (TR); Ercan SIMSEK, Nilüfer, Bursa (TR); Gary Arlen ANDERSON, Brookings, SD (US); Erkan YASLIOGLU, Nilüfer, Bursa (TR); Mete YILMAZ, Yildirim, Bursa (TR); Yahya ULUSOY, Nilüfer, Bursa (TR); Xufei YANG, Brookings, SD (US); Augustina OSABUTEY, Minneapolis, MN (US)

(21) Appl. No.: 18/577,417

(22) PCT Filed: Dec. 7, 2022
(86) PCT No.: PCT/TR2022/051445

§ 371 Date: Jan. 8, 2024

(30) Foreign Application Priority Data
Nov. 28, 2022 (TR) 2022/018039

(51) Int. Cl. C12M 1/00; C12M 1/34

(52) U.S. Cl. CPC C12M 21/02; C12M 29/06; C12M 31/02; C12M 41/34; C12M 45/04

(57) ABSTRACT

Disclosed is a photobioreactor system that reduces pollutant gases, mainly ammonia and carbon dioxide, released in poultry houses with microalgae and reduces dust and particulate matter in the internal environment of the poultry house, and also creates a brighter and more spacious environment for animals in the internal environment, while creating an insulation effect on the exterior of the poultry house by integrating into the exterior wall.

### TECHNICAL FIELD

The invention relates to a photobioreactor system for use in poultry houses.

In particular, the invention relates to a photobioreactor system that reduces pollutant gases, mainly ammonia and carbon dioxide, released in poultry houses (chicken coop) by microalgae and reduces dust and particulate matter in the internal environment of the house by means of an organic oil bath filter, and also creates a brighter and more spacious environment for animals in the internal environment, while creating an insulation effect on the exterior of the house by integrating into the exterior wall.

### Known State of the Art

The source of ammonia gas in poultry houses is chicken manure, while carbon dioxide gas is released by the respiration of chickens. Ammonia gas concentrations in poultry houses vary according to the poultry house structure system, capacity, number of animals and food content. Ammonia gas increases especially in cases of high protein value in food content and high number of animals. When the pollutant gas concentrations released from poultry farms are examined; ammonia gas is in the range of 1-48.3 ppm, carbon dioxide gas is 210-4300 ppm, H2S gas is in the range of 1.53-5.94 ppm and methane gas is in the range of 0-2 ppm. (Cheng et al., 2011; Guiziou and Belin, 2005; Borso and Chiumenti, 1999; Redwine et al., 2002; Wheeler et al., 2003; Kocaman et al., 2006; Liang et al., 2005; Radon et al., 2002; Hörnig et al., 2004; Mihina et al., 2012; Okoli et al., 2004;)

There are various studies in the literature on the reduction of pollutant gases from animal housing. Two different categories of methods, namely “source-based” and “end-of-pipe”. are generally used to reduce emissions from poultry houses (Heyden et al., 2015). “Source Based” methods are practices on manure management, storage and removal of manure (Clanton et al., 2001; Shah et al., 2014), regulation of animal feed content (Jacob et al., 2000; Erickson et al., 2000; James et al., 1999). “End-of-Pipe” techniques are applications using chemical, biological, biofilter and air scrubbers (Melse and Ogink, 2005; Lemay et al., 2009; Jacobsen, 2011). Studies have shown that chemical air-scrubber systems are more effective in mitigating ammonia gas, while biological air-scrubber and biofilter systems are effective in reducing odor (Heyden et al., 2015). When the ammonia concentration in the poultry house indoor gas rises above 35 ppm, the effectiveness of air scrubber systems decreases significantly (Lahav et al., 2008; Melse and Ogink, 2005).

There are many applications of biofilter systems to reduce pollutant gases and odor released from poultry houses. In these systems, mitigation efficiency of 80% in ammonia gas and approximately 95% in hydrogen sulfide gas has been achieved. However, for these systems to work effectively, the indoor humidity and temperature of the poultry house should be controlled and kept within certain ranges (Dalolio et al., 2015). The disadvantage of biofilter systems is that increasing the ventilation efficiency in temperate regions, especially in summer, causes the ammonia concentration to drop below 10 ppm, which reduces the effectiveness of the biofilter system (Lahav et al., 2008).

In poultry farming, dust and particulate matter concentrations in the indoor air of poultry houses are high and have a negative impact on the environment, human and animal health. Dust and particulate matter include a large component of manure material, as well as food, dandruff (skin material), feather material and microorganisms. In addition, odor components from the indoor air of the poultry house are also carried with dust and particulate matter. This causes odor pollution in the areas where the poultry houses are located. Therefore, when the amount of dust emitted from poultry houses is reduced, odor can be indirectly reduced (Guo et al., 2019). Methods such as cleaning ventilation inlet and outlet points (Li et al., 2019) and the design of the housing system are used to reduce the emission of particulate matter in poultry houses. Methods such as orienting the poultry houses according to the prevailing wind direction and arranging the location of building entrances and exits accordingly are methods used to keep the indoor and outdoor air clean. In mechanically ventilated poultry houses, there are applications to reduce both pollutant gases and particulate matter with biological air mixers (Bio-scrubber) at the exit point of the fans on the side walls. However, Aarnink et al. (2011) studies higher microbial concentrations was observed when biological air mixers were used at the point of ventilation. Ogink and Aarnink (2011) developed a Manure Drying Tunnel (MDT) to reduce PM emissions from poultry farming facilities. The studies showed that the MDT system significantly reduced PM concentrations in the indoor air of the poultry house. However, the major disadvantage of this system is that while the MDT system filters the particulate matter in the dry air inside the tunnel, it also emits unwanted pollutant gas and odor components to the outdoor environment.

In poultry houses, indoor environmental conditions directly affect production efficiency. In environmentally controlled poultry houses for chickens, it is very important to provide the necessary environmental conditions for light, heating, cooling and ventilation conditions, animal welfare, meat and egg production and quality. When chickens are exposed to cold temperatures, they eat more feed to maintain their body temperature and the feed they consume to keep warm cannot be converted into meat. On the other hand, at high temperatures, energy is also consumed by cooling systems to keep the animals cool.

Due to the increasing energy crises in recent years, the interest in the energy consumed in poultry is increasing. In poultry houses, energy is consumed for internal climate settings such as heating, cooling, ventilation, lighting, humidity control and for the operation of production equipment such as nutrition, sanitation and egg production. Of the total energy consumed in poultry houses, 84% is for heating, 7% for ventilation, 6% for lighting and 3% for other uses. Of the total electricity consumption, 45% is for ventilation, 37% for lighting, 13% for motors and water pumps, and 5% for other uses (Corkery et al., 2013).

Looking at the energy consumption for heating in poultry houses, it was observed that the annual LPG consumption in a well-insulated poultry house was 188000 kWh, while the LPG consumption in a less insulated poultry house was 214000 kWh (Baxevanou et al., 2017). Küçüktopçu and Cemek (2018) examined the effect of XPS and EPS insulation materials on energy savings in poultry houses and stated that energy savings up to 69% for XPS and 70% for EPS were achieved.

Lighting in poultry houses plays an important role in chicken welfare and productivity, and indoor lighting is provided by artificial means in a controlled environment to reduce chicken aggression and optimize production. The lighting program commonly used in poultry houses consists of 16 hours of light and 8 hours of darkness. Continuous lighting with a minimum light intensity of 20 lux is recommended in the post-hatching phase to help chicks adapt to the environment and nutrition. Studies have reported that chickens raised under yellow, green and blue light sources have better body weight than those raised under red and orange light sources (Oloyo and Ojerinde, 2018).

As mentioned above, studies on various emission reduction methods have been put forward in order to reduce the effects of pollutant gases released from livestock enterprises on human health and the environment. When we look at the poultry farms operating in our country, they create a welfare environment for animals with mechanical or natural ventilation methods. However, it is seen that they do not make any application on preventing or reducing the odor and pollution of the air discharged from the internal environment of the poultry house. The fact that the emission reduction methods developed with scientific studies and applied in developed countries are not sustainable systems besides their high costs negatively affects their applicability in our country. In this case, the question arises whether a more economical and sustainable method can be used by using the pollutant gases released from animal shelters in the production of microalgae, which is the renewable raw material source of recent years.

In line with the increasing need for alternative energy sources in the world in recent years, microalgae have emerged as one of the renewable raw material sources. As a renewable resource, microalgae have an important role in reducing greenhouse gas emissions, especially in biofuel production. The fact that the growth rate of microalgae is very high and therefore their biomass yields are very high, that they do not need arable land for their production, that they contain high oil and that they use carbon dioxide and other greenhouse gases, mainly from waste flue gases, for all these, shows that they are an economically sustainable energy source (Soydemir, 2016). Today, there are very few studies on the use of microalgae in reducing greenhouse gases. In particular, the effect of ammonia and carbon dioxide gas, which are pollutant gases released from animal production, on algal growth has been examined. However, these studies were conducted under laboratory conditions using pure ammonia and carbon dioxide gases (Kang et al., 2013; Kang and Wen, 2015). The closest of these studies to shelter conditions is the study of Li et al. (2017) on the reduction of gases and particulate matter released from fertilizer stored in a box with microalgae.

However, as can be understood from the literature, the concentrations of indoor gases and the growth of microalgae differ according to the environmental conditions of the environment. Therefore, in the known state of the art, there is a need for a system that enables the reduction of pollutant gases, especially ammonia and carbon dioxide, emitted in poultry houses with microalgae.

The document numbered US20120279119 can be shown as an example of the state of the art in the research conducted in the literature. The said document relates to apparatus and methods for producing biofuels. In the invention in question, it is mentioned that carbon dioxide-rich air can be infused into water containing algae by bubbling technique and a blower that transmits air containing polluted gases to the environment which is containing algae. However, the invention does not include the reduction of pollutants such as dust, PM, CO2, NH3, H2S in indoor air by means of a photobioreactor system. In addition, the invention does not include a structure that reduces dust and particulate matter with an organic oil bath air filter and at the same time provides an insulation effect in maintaining the indoor temperature of the poultry house.

The document numbered US20080178739 can be shown as another example of the state of the art. The document discloses processes for processing gases such as flue gases with photobioreactors and for operating and using photobioreactors for biomass production. The use of said photobioreactor system as part of a fuel production system and/or a gas treatment process comprises at least partially removing certain undesirable contaminants from a gas stream. In the present invention, however, the contaminated gas is not transferred into water but into a space above the water.

As a result, the existence of the above problems and the inadequacy of existing solutions necessitated a development in the relevant technical field.

### Objective of the Invention

The present invention relates to a photobioreactor system for reducing ammonia and carbon dioxide gases in poultry houses, which eliminates the above-mentioned disadvantages and brings new advantages to the related technical field.

The main objective of the invention is to reduce the pollutant gases (NH3, CO2, H2S) emitted from poultry houses with a photobioreactor (PBR) system in which microalgae is grown.

The objective of the invention is to ensure that the dust and particulate matter released from the poultry houses are cleaned from the indoor air of the poultry house supplied to the PBR system with an organic oil bath air filter.

Another objective of the invention is to utilize the harvested biomass as biofuel and chicken feed.

Another objective of the invention is to keep the indoor air clean by circulating the output air of the photobioreactor system (rich in oxygen) back into the indoor environment of the shelter with a heat exchanger or to heat the cold air coming into the indoor environment in cold weather.

Another objective of the invention is to increase the egg and meat yields of chickens by cleaning the indoor air.

Another objective of the invention is to maintain the indoor temperature of the poultry house by means of a photobioreactor system integrated into the exterior of the poultry house and to create an insulation effect in the poultry house.

Another objective of the invention is to enable the photobioreactor system to be built on the exterior of the poultry house instead of a wall and to create a dimly lit environment in the interior of the poultry house due to the high light transmittance of the photobioreactor tanks made of transparent acrylic glass.

Another objective of the invention is to reduce the pollutant gases released from poultry houses with an economical and sustainable system.

Another objective of the invention is to produce raw material for the organic oil bath air filter to be used in the system with the biomass to be obtained in photobioreactors. Another objective of the invention is to enable the photobioreactor system to be used as a biological insulation material for poultry house structures.

Another objective of the invention is to integrate the photobioreactor system into the exterior walls of the poultry house, to obtain a bright indoor environment by reflecting the daylight coming on the photobioreactors to the indoor environment and to provide an economical and environmentally friendly lighting for chickens in the poultry house by changing the color of the light reflected from the photobioreactor to the indoor environment according to the color of the algae species to be used in the system.

Another objective of the invention is to contribute to the measures taken for global warming and climate change.

Another objective of the invention is to contribute to scientific studies in the fields of sustainable agriculture, renewable energy and energy storage and sustainable environmental management technologies, ecosystems and sustainable built environment.

Another objective of the invention is to minimize the emission of greenhouse gases, to create animal welfare conditions in shelters and to use the biomass obtained as biofuel and animal feed.

Another objective of the invention is to ensure that the raw material of the oil to be used in the organic oil bath air filter is made with the oil obtained from the microalgae biomass produced in the system, thus ensuring the sustainability of the system.

Another objective of the invention is to keep the indoor temperature in the poultry houses warm in winter and cool in summer, thereby reducing the energy cost used by the enterprise for heating and cooling.

In order to fulfill all the purposes stated above and which may arise from the detailed explanation, the invention is a photobioreactor system for the reduction of pollutant gases, particularly ammonia and carbon dioxide, released from poultry houses;
a gas inlet line connected to the ventilation fans from which the poultry house indoor air comes out and which allows the poultry house indoor air to enter the photobioreactor system,
an organic oil bath filter system located on the gas inlet line that prevents the entry of dust and particulate matter in the indoor air of the poultry house into the microalgae culture environment,
a pump system located on the gas inlet line, which allows the poultry house indoor air to be vacuumed from the gas inlet line and transferred to the photobioreactor system,
a photobioreactor tank that is adapted to the exterior walls of the poultry house and provides cleaning of pollutant gases in the indoor air of the poultry house by means of microalgae cultures drawn by the pump system and at the same time provides insulation and lighting on the exterior of the poultry house,
an air diffuser located at the bottom of the photobioreactor tank, which transfers the poultry house indoor air drawn by the pump system to the photobioreactor tank and at the same time ensures the mixing of the culture medium in order to keep the microalgae homogeneous in the photobioreactor tank,
an air outlet line that allows the air cleaned as a result of photosynthesis of microalgae cultures in the photobioreactor tank to be delivered back to the indoor environment of the poultry house.

The structural and characteristic features and all the advantages of the invention will be more clearly understood by means of the figures given below and the detailed description written by making references to these figures. Therefore, the evaluation should be made by taking these figures and the detailed description into consideration.

### FIGURES TO HELP UNDERSTAND THE INVENTION

FIG. 1: Schematic view of the inventive photobioreactor system.

### DESCRIPTION OF PART REFERENCES

10. Gas inlet line
20. First gas sensor
30. Organic oil bath filter system
40. Pump system
50. Flowmeter
60. Photobioreactor tank
70. Air diffuser
80. Air outlet line
90. Second gas sensor

### DETAILED DESCRIPTION OF THE INVENTION

In this detailed description, the preferred alternatives of the inventive photobioreactor system are described only for the purpose of a better understanding of the subject matter and in a non-limiting manner.

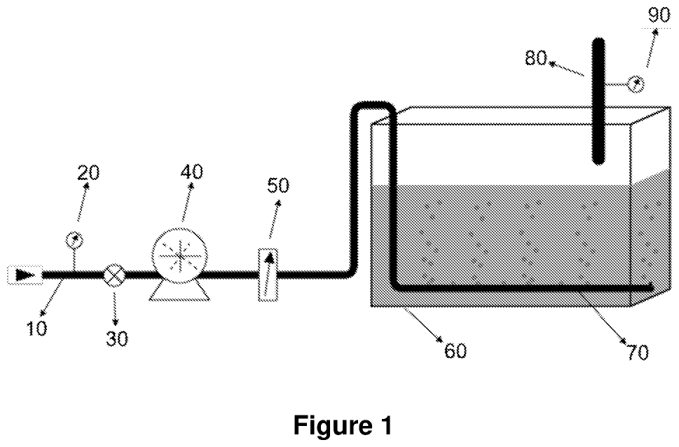

FIG. 1 shows a schematic view of the inventive photobioreactor system. Accordingly, the photobioreactor system is in its most basic form; gas inlet line (10) that allows the poultry house indoor air to enter the photobioreactor system, first gas sensor (20) that determines the amount of ammonia and carbon dioxide gas in the poultry house indoor air on the gas inlet line (10), organic oil bath filter system (30) that prevents dust and particulate matter in the poultry house indoor air from entering the microalgae culture medium on the gas inlet line (10), pump system (40) that provides vacuuming of the poultry house indoor air on the gas inlet line (10), flowmeter (50) that adjusts the flow rate of the vacuumed poultry house indoor air on the gas inlet line (10), photobioreactor tank (60) that is adapted to the exterior walls of the poultry house and provides cleaning of the pollutant gases in the poultry house indoor air drawn by the pump system (40) by means of microalgae cultures and also provides insulation and illumination on the exterior of the poultry house, air diffuser (70) located at the bottom of the photobioreactor tank (60), which transfers the indoor air of the poultry house drawn by the pump system (40) to the photobioreactor tank (60) and at the same time ensures the mixing of the culture medium in order to keep the microalgae homogeneous in the photobioreactor tank (60), the air outlet line (80) that allows the air cleaned as a result of photosynthesis of microalgae cultures in the photobioreactor tank (60) to be delivered back to the indoor environment of the poultry house, and a second gas sensor (90) located on the air outlet line (80) that measures how much pollutant gas remains in the polluted air cleaned in the photobioreactor tank (60).

In the photobioreactor system subject to the invention, it is provided that the indoor air in the poultry house enters the photobioreactor system through the gas inlet line (10) to be cleaned. For this purpose, the gas inlet line (10) is connected to the ventilation fans from which the indoor air of the poultry house comes out.

At least one first gas sensor (20) is provided on the gas inlet line (10), which determines the amount of ammonia and carbon dioxide gas in the indoor air of the house. The scale of the photobioreactor system is adjusted according to the amount of gas determined by the first gas sensors (20).

On the gas inlet line (10), there is an organic oil bath filter system (30) that prevents dust and particulate matter in the indoor air of the poultry house from entering the microalgae culture environment.

There is oil at the bottom of the oil bath filter system (30) and particles such as dust, dirt, sand in the air passing through the oil bath filter system (30) fall into the oil bath. At least one wire mesh screen at the outlet of the oil bath filter system (30) traps the oil and prevents it from entering the photobioreactor tank (60). In addition, dust and particulate matter in the air are also captured by the said wire mesh screen and separated from the air. The raw material of the oil used in the oil bath filter system (30) is made with the oil obtained from the microalgae biomass produced in the photobioreactor system. In this way, the sustainability of the photobioreactor system is ensured.

The gas inlet line (10) is provided with a pump system (40) for vacuuming the indoor air of the poultry house. Said pump system (40) is preferably a pump, which in an alternative embodiment of the invention may be a blower or a vacuum device. The oil bath filter system (30) is positioned in front of the pump system (40) and the oil bath filter system (30) prevents the transportation of dust and particulate matter in the indoor air of the poultry house.

On the gas inlet line (10), there is a flowmeter (50) that adjusts the flow rate of the vacuumed poultry house indoor air. The amount of air to be supplied according to the microalgae culture density in the photobioreactor tank (60) is adjusted by the flowmeter (50).

The photobioreactor tank (60), which is adapted to the exterior walls of the poultry house and contains microalgae cultures, is made of transparent acrylic glass. In this way, it is adapted to the exterior walls of the poultry house and provides thermal insulation and lighting on the exterior of the poultry house. The photobioreactor tank (60) provides cleaning of pollutant gases in the indoor air of the poultry house by means of microalgae cultures drawn by the pump system (40).

At the bottom of the photobioreactor tank (60), there is an air diffuser (70) that transfers the poultry house indoor air drawn by the pump system (40) to the photobioreactor tank (60) and at the same time mixes the culture medium in order to keep the microalgae homogeneous in the photobioreactor tank (60).

The air, which is cleaned as a result of photosynthesis of microalgae cultures in the photobioreactor tank (60), is transferred back to the indoor environment of the poultry house through the air outlet line (80) passing through the photobioreactor tank (60).

On the air outlet line (80), there is at least one second gas sensor (90) that measures how much pollutant gas remains in the polluted air cleaned in the photobioreactor tank (60). The extent to which ammonia and carbon dioxide gases have been reduced in the photobioreactor tank (60) is determined by gas measurements from the second gas sensor (90).

### The Operating Principle of the Inventive Photobioreactor System is as Follows;

The ventilation fans from which the poultry house indoor air is extracted are connected to the gas inlet line (10) and the indoor air is drawn by the pump system (40). The amount of pollutant gases in the poultry house indoor air is measured by first gas sensors (20).

The oil bath filter system (30) in front of the vacuum pump (20) prevents the transportation of dust and particulate matter in the indoor air of the poultry house. The amount of air to be given according to the microalgae density in the photobioreactor tank (60) is adjusted by flowmeter (50).

The polluted air drawn by the pump system (40) is transferred into the photobioreactor tank (60) by air diffusers (70). Ammonia and carbon dioxide gases in the polluted air are reduced by microalgae in the photobioreactor tank (60).

The amount of reduction of ammonia and carbon dioxide gases is determined by measurements made with the second gas sensors (90) located in the air outlet line (80). During the poultry house indoor air purification, the photobioreactor tank (60) provides heat insulation and illumination on the outside of the poultry house.

1. A photobioreactor system that enables the reduction of pollutant gases, mainly ammonia and carbon dioxide, released from poultry houses, the system comprising:
a gas inlet line that connects to the ventilation fans from which the indoor air of a poultry house exits and allows the indoor air of the poultry house to enter the photobioreactor system;
an organic oil bath filter system located on the gas inlet line, which prevents the entry of dust and particulate matter in the indoor air of the poultry house into the microalgae culture environment;
a pump system located on the gas inlet line, which allows the poultry house indoor air to be vacuumed from the gas inlet line and transferred to the photobioreactor system;
a photobioreactor tank which is adapted to the exterior walls of the poultry house and provides insulation and illumination of the exterior of the poultry house, as well as cleaning of pollutant gases in the indoor air of the poultry house by means of microalgae cultures, which are drawn by the pump system;
an air diffuser located at the bottom of the photobioreactor tank, which transfers the poultry house indoor air drawn by the pump system to the photobioreactor tank and at the same time ensures the mixing of the culture medium in order to keep the microalgae homogeneous in the photobioreactor tank; and,
an air outlet line that allows the air cleaned as a result of photosynthesis of microalgae cultures in the photobioreactor tank to be delivered back to the indoor environment of the poultry house.

2. The photobioreactor system according to claim 1, comprising; at least one first gas sensor located on said gas inlet line and determining the amount of ammonia and carbon dioxide gas in the indoor air of the house.

3. The photobioreactor system according to claim 1, comprising; a flowmeter located on said gas inlet line and regulating the flow rate of the poultry house indoor air vacuumed by a pump system.

4. The photobioreactor system according to claim 1, wherein said photobioreactor tank is made of transparent acrylic glass.

5. The photobioreactor system according to claim 1, comprising; at least one second gas sensor located on said air outlet line and measuring how much contaminant gas remains in the contaminated air cleaned in the photobioreactor tank.

6. The photobioreactor system according to claim 1, comprising; an oil located at the bottom of said oil bath filter system and obtained from microalgae biomass produced in the photobioreactor system for separating particles such as dust, dirt and sand in the air passing through the oil bath filter system.

7. The photobioreactor system according to claim 1, characterized comprising; at least one wire mesh screen located at the outlet of said oil bath filter system and preventing oil from being trapped at the bottom of said oil bath filter system and entering the photobioreactor tank.

8. The photobioreactor system according to claim 1, wherein said pump system is a pump, blower or vacuum means.
